(19) United States
(12) Patent Application Publication
(10) Pub. No.: US 2024/0287090 A1
(43) Pub. Date: Aug. 29, 2024

(54) SOLID STATE FORMS OF RELUGOLIX

(71) Applicant: Cipla Limited, Mumbai (IN)

(72) Inventors: Arijit DAS, Porvorim (IN); Ramanaiah CHENNURU, Nellore (IN); Anjaneyaraju INDUKURI, Hyderabad (IN); Lakkireddy PULLAREDDY, Kadapa (IN); Pyla Kranthi TEJA, Visakhapatnam (IN)

(21) Appl. No.: 18/691,556

(22) PCT Filed: Aug. 17, 2022
(86) PCT No.: PCT/IN2022/050742

§ 371 Date: Mar. 13, 2024

(30) Foreign Application Priority Data
Sep. 15, 2021 (IN) 202121041687

(51) Int. Cl. C07D 495/04; A61K 31/519

(52) U.S. Cl. CPC C07D 495/04; A61K 31/519

(57) ABSTRACT

The present invention relates to solid state forms of Gonadotropin-Releasing Hormone Receptor (GnRH) antagonist of Formula (I) and a pharmaceutically acceptable salt thereof, namely 1-(4-(1-(2,6-difluorobenzyl)-5-((dimethylamino)methyl)-3-(6-methoxy pyridazin-3-yl-2,4-dioxo-1,2,3,4-tetrahydrothieno(2,3-d)pyrimidin-6-yl)phenyl)-3-methoxyurea, and its pharmaceutically acceptable salts thereof, methods of their preparation, pharmaceutical compositions thereof and methods of their use.

### FIELD OF THE INVENTION

The present invention relates to solid state forms of the Gonadotropin-Releasing Hormone Receptor (GnRH) antagonist of Formula (I) or and pharmaceutically acceptable salts thereof, process for preparation thereof and pharmaceutical composition comprising solid state forms thereof.

### BACKGROUND OF THE INVENTION

Relugolix, is a once a daily selective antagonist of Gonadotropin-Releasing Hormone Receptor, under development for the treatment of certain pathologies, production of testicular testosterone-which stimulates prostate cancer growth and ovarian estradiol—which stimulates endometriosis and uterine leiomyoma.

Relugolix is chemically termed as 1-(4-(1-(2,6-difluorobenzyl)-5-((dimethylamino)methyl)-3-(6-methoxypyridazin-3-yl)-2,4-dioxo-1,2,3,4-tetrahydrothieno(2,3-d)pyrimidin-6-yl)phenyl)-3-methoxyurea, having the following chemical structure:

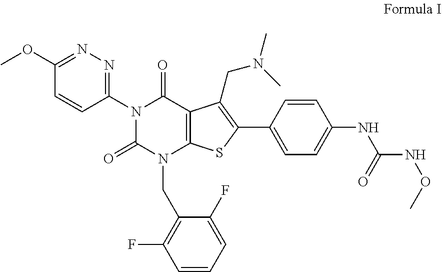 [SMILES: CONC(=O)Nc1ccc(-c2sc3c(c2CN(C)C)c(=O)n(-c2ccc(OC)nn2)c(=O)n3Cc2c(F)cccc2F)cc1]

The earliest known synthesis of Relugolix and pharmaceutically acceptable salts thereof, is described in the U.S. Pat. No. 7,300,935 B2.

U.S. Pat. No. 9,758,528 B2 discloses an alternate process to prepare Relugolix.

In addition, J. Med. Chem., 2011, 54 (14), pp. 4998-5012, refers to pharmacological and chemical aspects of Relugolix.

Prior art suggests that Relugolix or a pharmaceutically acceptable salt thereof exists in the various polymorphic forms.

U.S. Pat. No. 10,464,945 B2 discloses processes and crystalline forms of Relugolix or a pharmaceutically acceptable salt thereof characterized by XRD. More specifically the patent covers crystalline form of a THF solvate of Relugolix or a pharmaceutically acceptable salt thereof.

US 2021/0017188 A1 patent application discloses crystalline forms F, G and H of Relugolix characterized by XRD and FTIR.

Although the above-mentioned patent applications describe various polymorphic forms of Relugolix and its salts thereof, it is a well-known fact that different salts and polymorphic forms of the same drug may have substantial differences in certain pharmaceutically important properties such as dissolution characteristics, bioavailability patterns, handling properties, solubility, flow characteristics and stability.

Further, different physical forms may have different particle size, hardness and glass transition temperatures.

Therefore, the development of new solid-state forms of a pharmaceutically useful compound provides a new opportunity to improve the performance characteristics of a pharmaceutical product.

These solid state forms exhibit distinct X-ray diffractogram, solid state ¹³C NMR spectrometry, infrared spectrometry. Further, these solid state forms may give rise to peculiar thermal behaviour which can be measured by melting point, thermo gravimetric analysis (TGA), differential scanning calorimetry (DSC). All these properties can be used to distinguish a particular solid state form from the other forms.

### OBJECT OF THE INVENTION

The object of the present invention is to provide novel solid state forms of Relugolix or pharmaceutically acceptable salts thereof.

Yet another object of the present invention is to provide a novel process for preparing the novel solid state forms of Relugolix or pharmaceutically acceptable salts thereof which is simple, economical and suitable for industrial scale-up.

Yet another object of the present invention is to provide a pharmaceutical composition comprising novel solid state forms of Relugolix or pharmaceutically acceptable salts and pharmaceutically acceptable carrier, diluent or excipients.

Yet another object of the present invention is to use pharmaceutical composition defined hereinabove for the treatment of certain pathologies, e.g., endometriosis, uterine leiomyoma, and prostate cancer.

### SUMMARY OF THE INVENTION

In a first embodiment, this invention is directed to novel solid state forms of Relugolix of Formula I or pharmaceutically acceptable salts thereof.

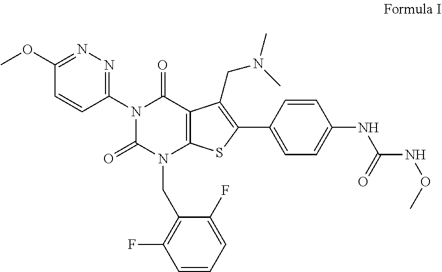 [SMILES: CONC(=O)Nc1ccc(-c2sc3c(c2CN(C)C)c(=O)n(-c2ccc(OC)nn2)c(=O)n3Cc2c(F)cccc2F)cc1]

More preferably, the invention encompasses crystalline forms of Relugolix of Formula I hereinafter referred to as Form-C1 and Form-C2.

The solid state forms of Relugolix may be in a pseudo polymorphic form. Accordingly, the pseudo polymorphs provided herein include hydrates and/or solvates.

The crystalline nature of solid state forms according to the present invention is characterized by X-ray powder diffraction pattern, DSC and TGA.

In a second embodiment, the invention encompasses processes for the preparation of solid state forms of Relugolix of Formula I or pharmaceutically acceptable salts thereof.

In a third embodiment, the present invention provides novel solid state forms of Relugolix of Formula I or pharmaceutically acceptable salts thereof, prepared according to the process described above, having a purity of more than about 95%, preferably at least 99%, more preferably at least 99.5% by HPLC.

In a fourth embodiment, the invention provides a pharmaceutical composition comprising novel solid state forms of Relugolix of Formula I or its pharmaceutically acceptable salts thereof, prepared by a process as described above, together with one or more pharmaceutically acceptable excipients. Such excipients are well known to those skilled in the art.

In a fifth embodiment, the pharmaceutical composition comprising novel solid state forms of Relugolix of Formula I or its pharmaceutically acceptable salts thereof can be formulated into variety of dosage forms which include solid dosage forms like tablets, powders, granulates, capsules, sachets, aggregates, suppositories, troches, and lozenges, as well as liquid syrups, suspensions, and elixirs.

In a sixth aspect, the invention provides a process for preparation of pharmaceutical composition comprising crystalline Form C2 of Relugolix which process comprises, combining crystalline Form C2 of Relugolix with one or more suitable pharmaceutical excipients.

In a seventh embodiment, the invention provides novel solid state forms of Relugolix of Formula I or its pharmaceutically acceptable salts thereof, prepared by a process as described above for use in the treatment of diseases caused by gonadotropin releasing hormone (e.g., endometriosis, uterine fibroid and prostate cancer), and can be used for the prophylaxis or treatment of the above-mentioned diseases.

In an eighth embodiment, the invention provides the use of novel solid state forms of Relugolix of Formula I or its pharmaceutically acceptable salts thereof, prepared by a process as described above, in the manufacture of a medicament for treating diseases caused by gonadotropin releasing hormone (e.g., endometriosis, uterine fibroid and prostate cancer).

In a ninth embodiment, the invention provides a method of treating diseases caused by gonadotropin releasing hormone (e.g., endometriosis, uterine fibroid and prostate cancer) in a patient in need of such treatment, which method comprises administering to the patient a therapeutically effective amount of novel solid state forms of Relugolix of Formula I or its pharmaceutically acceptable salts thereof, prepared by a process as described above.

### BRIEF DESCRIPTION OF DRAWINGS

FIG. 1 shows an X-ray powder diffraction pattern (XRPD) of Form-C1 of Relugolix

FIG. 2 shows a thermogravimetric analysis (TGA) of Form-C1 of Relugolix

FIG. 3 shows the thermoanalysis and determination of the melting point (DSC) of Form-C1 of Relugolix

FIG. 4 shows an X-ray powder diffraction pattern (XRPD) of Form-C2 of Relugolix

FIG. 5 shows a thermogravimetric analysis (TGA) of Form-C2 of Relugolix

FIG. 6 shows the thermoanalysis and determination of the melting point (DSC) of Form-C2 of Relugolix

### DETAILED DESCRIPTION OF THE INVENTION

Unless defined otherwise, technical and scientific terms used herein have the same meaning as commonly understood by one of ordinary skill in the art to which this invention belongs, and are consistent with:

### Definitions

“Polymorph”, as used herein, refers to the occurrence of different crystalline forms of a compound. Crystalline forms have different arrangements and/or conformations of the molecule in the crystal lattice. Solvates are crystal forms containing either stoichiometric or nonstoichiometric amounts of a solvent. If the incorporated solvent is water, the solvate is

commonly known as a hydrate. Therefore, a single compound may give rise to a variety of polymorphic forms where each form has different and distinct physical properties, such as solubility profiles, melting point temperatures, hygroscopicity, particle shape, density, flowability, compactability and/or x-ray diffraction peaks.

The solubility of each polymorph may vary, thus, identifying the existence of pharmaceutical polymorphs is essential for providing pharmaceuticals with predictable solubility profiles. It is desirable to investigate all solid state forms of a drug, including all polymorphic forms, and to determine the stability, dissolution and flow properties of each polymorphic form.

A crystalline solid substance is characterized by a regular three-dimensional arrangement of atoms due to which they have well defined geometrical shape. On the other hand, amorphous solid substances do not exhibit this arrangement. Thus, compared to crystalline solid substances, amorphous solid substances have a different internal structure and a larger surface area, and therefore they exhibit a higher solubility. If the solubility and bioavailability of pharmaceutically active substances needs to be increased, they are preferably prepared in an amorphous form.

As used herein, the term “PXRD” refers to powder X-ray diffraction, the term “IR” refers to infrared, the term “NMR” refers to nuclear magnetic resonance, the term “TGA” refers to thermogravimetric analysis, the term “DSC” refers to differential scanning calorimetry and the term “DVS” refers to dynamic vapour sorption isotherm.

As used herein, the term “substantially the same X-ray powder diffraction pattern” is understood to mean that those X-ray powder diffraction patterns having diffraction peaks with 20 values within ±0.2° of the diffraction pattern referred to herein are within the scope of the referred to diffraction pattern.

As used herein, the term “solvate” refers to an association or complex of one or more solvent molecules and a compound of the invention. Such solvents for the invention may not interfere with the biological activity of the solute. Typically, the solvent used is a pharmaceutically acceptable solvent. Examples of solvents that form solvates include, but are not limited to, C1-C4 alcohol solvents such as isopropanol, ethanol, methanol, butanol, aromatic alcohols such as benzyl alcohol, phenethyl alcohol; esters such as methyl benzoate, methyl acetate, ethyl acetate; nitriles such as acetonitrile; chlorinated solvents such as dichloromethane; ethers such as tetrahydrofuran (THF), diethyl ether, dimethyl ether and ketones such as acetone, other than water at levels of more than 1%.

The solvate can be isolated either as an amorphous form or in a crystalline form, preferably in crystalline form.

The solvate can be further isolated either in anhydrous form or hydrated form.

As used herein, the term “hydrate” refers to the complex where the solvent molecule is water. The skilled person will appreciate that the water molecules are absorbed, adsorbed or contained within a crystal lattice of the solid compounds, usually in defined stoichiometric ratio. The notation for a hydrated compound may be. nH₂O, where n is the number of water molecules per formula unit of the compound. For example, in a hemihydrate, n is 0.5; in a monohydrate n is one; in a sesquihydrate, n is 1.5; in a dihydrate, n is 2; and so on.

In comparison to the restricted stoichiometric hydrates, non-stoichiometric hydrates can vary in water content without major change in their crystal structure. The amount of water in the crystal lattice only depends on the partial pressure of water in the surrounding atmosphere.

Structurally, non-stoichiometric hydrates normally show channels or networks, through which the water molecules can diffuse. Depending on how the water is arranged inside the crystals, they are classified as isolated hydrates, channel hydrates and ion associated hydrates.

The novel polymorphs of the present invention may be isolated in pseudo polymorphic form as a solvate optionally in hydrated form, or as a non-hydrated solvate.

As polymorphic forms are reliably characterized by peak positions in the X-ray diffractogram, the polymorphs of the present invention have been characterized by powder X-ray diffraction spectroscopy which produces a fingerprint of the crystalline form and is able to distinguish it from all other crystalline and amorphous forms of Relugolix. Measurements of 2θ values are accurate to within ±0.2 degrees. All the powder diffraction patterns were measured on a PANalytical X'Pert³ X-ray powder diffractometer with a copper-K-α radiation source.

Seeding is a technique of using a single crystal or more to induce the formation of more crystals from a mixture, solution, or suspension. A seeding amount is the amount of material that, when added to a mixture, solution, or suspension, is able to cause the formation of the desired form of a compound. While in theory, this amount can be very small, in practice, a larger amount is used. This amount can be any amount that can be reasonably handled and is sufficient to cause the formation of the desired form of a compound. As a non-limiting example, amounts of 0.0001% to 50% wt/wt of the seeding compound based on a reference compound can be used as a seeding amount.

The term “substantially pure” means a particular form substantially free of other forms.

The invention will now be described in detail in connection with certain preferred and optional embodiments, so that various aspects thereof may be more fully understood and appreciated.

In a first embodiment, the invention provides a novel crystalline form of Relugolix of Formula I, which forms the first aspect of the present invention. The crystalline form is referred to as “Form-C1”, provided according to the example 1.

Accordingly, the crystalline Relugolix Form-C1 is characterized by having an X-ray powder diffraction spectrum comprising peaks at 7.09, 8.23, 8.86, 16.56, and 21.28±0.2° 2θ.

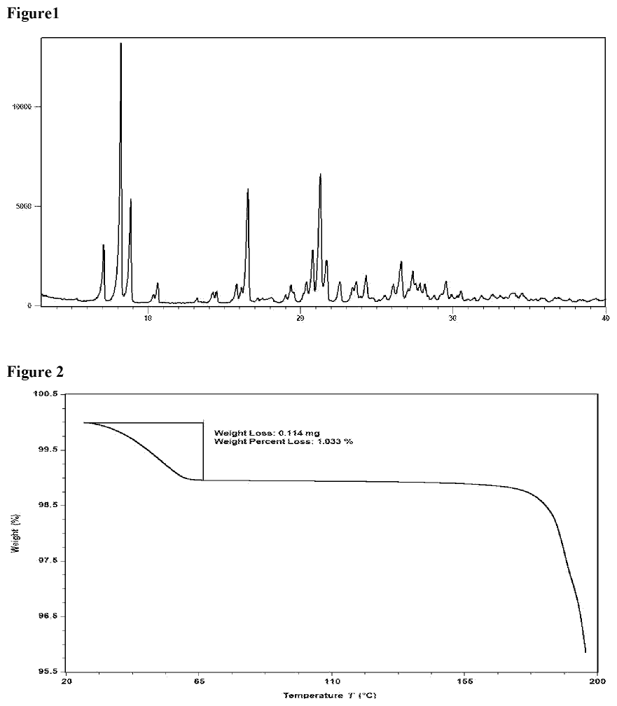

In another embodiment, crystalline Form-C1 of Relugolix of the present invention is characterized by having an X-ray powder diffraction spectrum as shown in FIG. 1.

The crystalline Form-C1 of Relugolix is characterized by having a TGA thermogram substantially as depicted in FIG. 2.

TGA data as shown in FIG. 2 indicated a weight loss of approximately 1.033% at temperature about 70° C.

Thus, the present invention provides a Crystalline Form C1 of Relugolix, characterized by data selected from one or more of the following:
a) an X-ray powder diffraction pattern substantially as depicted in FIG. 1;
b) an X-ray powder diffraction spectrum comprising peaks at 7.09, 8.23, 8.86, 16.56, and 21.28±0.2° 2θ;
c) a TGA thermogram substantially as depicted in FIG. 2;
d) TGA thermogram characterized by a weight loss of approximately 1.033% at temperature about 70° C.; and
e) combinations of the data, as in a) to d).

The X-ray powder diffraction spectrum of crystalline Relugolix Form-C1 is characterized by having further peaks at 10.63, 22.57 and 26.59±0.2° 2θ.

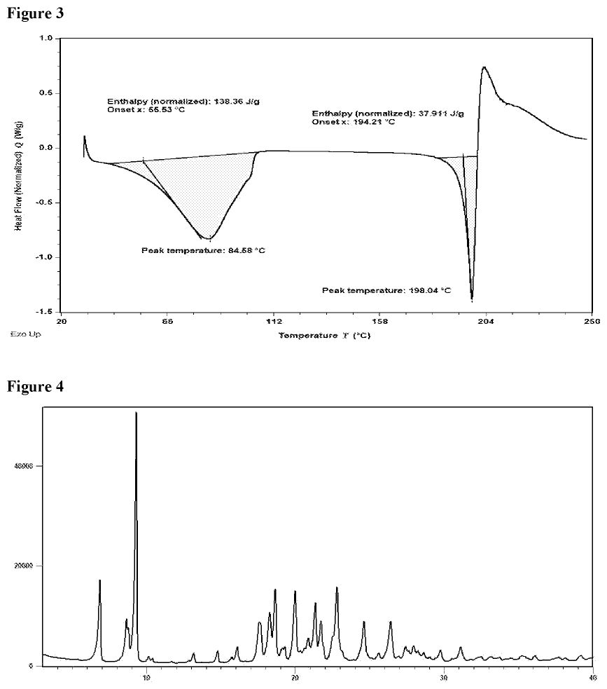

The crystalline Form-C1 of Relugolix is further characterized by DSC thermogram as shown in FIG. 3.

The DSC plot for the sample according to FIG. 3 shows two endotherm peaks one melting with an onset at 55.53±5° C., a peak maximum at 84.58±5° C. and second one melting with an onset at 194.21±5° C., a peak maximum at 198.04±5° C.

In another aspect of the invention, there is proved process for preparation of the crystalline Form-C1 of Relugolix, which process comprises;
a. Dissolving Relugolix in a suitable solvent or solvent mixture thereof at a temperature of about 0° C. to about reflux temperature of the solvent used;
b. Mixing the solution of step a) with one or more suitable anti-solvents at a temperature of about −10° C. to about 80° C.;
c. Stirring for a sufficient time; and
d. Isolating crystalline Form-C1 of Relugolix.

In anti-solvent crystallisation, the crystal form is obtained by the addition of an antisolvent to a solvent solution which induces crystallisation, followed by a filtration step. An anti-solvent crystallization technique is advantageous for improving solubility, dissolution and bioavailability of drugs with poor aqueous solubility. This method has an ability to change the solid-state properties of pharmaceutical substances including the modification of crystal formation and particle size distributions, preparation of nanoparticles or micro particles for poorly water-soluble drugs.

The Relugolix base used for the preparation of crystalline form C1 as in the above process, as well as for the following processes, may be in any polymorphic form or in a mixture of any polymorphic forms such as hydrated, solvated, non-solvated or mixture of hydrated, solvated or non-solvated forms thereof.

The Relugolix base as used in any of the processes described in the present application may be prepared according to the processes reported in U.S. Pat. No. 7,300,935, which is incorporated herein by reference.

Suitable solvent used in the process, as well as for the following processes is selected from, but not limited to, the group comprising of aprotic polar solvents, ethers, aromatic hydrocarbons, aliphatic hydrocarbons, mixtures of one or more organic solvents, wherein the organic solvent is preferably selected from the group comprising polar aprotic solvents such as N,N-dimethylacetamide (DMA), dimethylformamide (DMF), dimethylsulfoxide (DMSO), N-methylpyrrolidone (NMP), sulfolane, diglyme, 1,4-dioxane and the like; ether solvents such as methyl/-butyl ether, diisoproyl ether, tetrahydrofuran (THF) and the like; ester solvents such as methyl acetate, ethyl acetate, isopropyl acetate and the like; nitrile solvents such as acetonitrile, propionitrile and the like, ketone solvents such as acetone, methyl isobutyl ketone and the like; an aromatic hydrocarbons such as toluene, xylene and the like; aliphatic hydrocarbon solvents such as hexane, heptane and the like; and mixtures thereof.

Preferably, Relugolix is dissolved in a suitable solvent at a temperature of about 0° C. to about reflux temperature of the solvent used, preferably about 10° C. to about 90° C., more preferably about 20° C. to about 80° C., most preferably about 30° C. to about 70° C.

Preferably, an anti-solvent is used, in which the Relugolix is insoluble, to precipitate crystalline form C1. The crystalline form C1 precipitates as a consequence of the change of super saturation caused by mixing the solution and the antisolvent. The antisolvent may be, but is not necessarily, (partially) miscible with pure water. It is also possible to use an antisolvent or mixture of antisolvents which will result in the formation of an emulsion after it/they are added to the solution. Particularly preferred antisolvents for the antisolvent crystallisation process according to the invention are organic solvents.

The antisolvent organic solvent is preferably selected from the group comprising of water, protic polar solvents or mixtures thereof wherein protic polar solvents is preferably selected from C1-C5 alcoholic solvent such as methanol, ethanol, isopropanol, n-butanol, t-butanol and the like; aromatic alcohols, and mixtures thereof.

Preferably, after the antisolvent addition, a stirring step is performed at a temperature of about −10° C. to about 80° C., preferably about 0° C. to about 60° C., more preferably about 10° C. to about 50° C.; preferably, for about 1 hour to about 80 hours, more preferably about 10 hours to about 75 hours, most preferably about 20 hours to about 72 hours.

The crystalline Form-C1 of Relugolix thus precipitated is easily isolated from the reaction mixture by filtration.

Preferably, the isolated crystalline Form-C1 of Relugolix is dried under reduced pressure at 25-60° C., preferably at 30-50° C.; for at about 1 hour to about 30 hours.

In a second embodiment, the invention provides a novel crystalline form of Relugolix of Formula I, which forms the second aspect of the present invention.

The crystalline form herein after is referred to as “Form-C2”.

The crystalline Form-C2 is characterized by having an X-ray powder diffraction spectrum comprising peaks at 6.87, 9.30, 18.63, 19.97 and 22.78±0.2° 2θ.

In another embodiment, the crystalline Form-C2 of Relugolix of the present invention is characterized by having an X-ray powder diffraction spectrum as shown in FIG. 4.

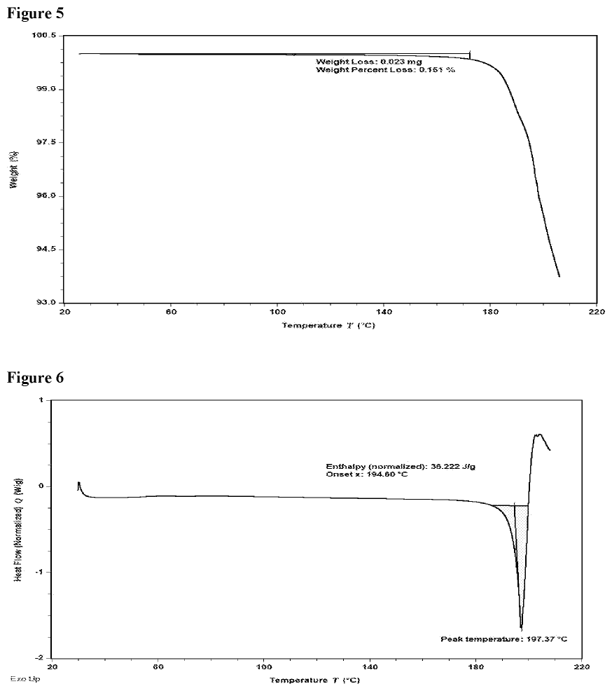

The crystalline Form-C2 of Relugolix is further characterized by having a TGA thermogram substantially as depicted in FIG. 5.

TGA data as shown in FIG. 5 indicated a weight loss of approximately 0.152% at temperature about 175° C.

Thus, the present invention provides a Crystalline Form C-2 of Relugolix, characterized by data selected from one or more of the following:
a) an X-ray powder diffraction pattern substantially as depicted in FIG. 4;
b) an X-ray powder diffraction spectrum comprising peaks at 6.87, 9.30, 18.63, 19.97 and 22.78±0.2° 2θ;
c) a TGA thermogram substantially as depicted in FIG. 5;
d) TGA thermogram characterised by a weight loss of approximately 0.152% at temperature about 175° C.; and
e) combinations of the above data.

The X-ray powder diffraction spectrum of form C2 is further characterized by having peaks at 16.09, 21.34, 24.61 and 26.40±0.2° 2θ.

The crystalline Form-C2 of Relugolix is further characterized by having a DSC thermogram as shown in FIG. 6.

The DSC plot for the crystalline Form-C2 of Relugolix shows a single endotherm peak melting with an onset at 194.60±5° C. and a peak maximum at 197.37±5° C.

Those skilled in the art would recognize that these crystal forms or amorphism can be identified by a variety of technical means, including, but not limited to infrared absorption spectroscopy (IR), melting point method, Nuclear magnetic resonance (NMR), Raman spectroscopy, dynamic vapor sorption (DVS), particle size, dissolution calorimetry, scanning electron microscopy (SEM), quantitative analysis, solubility, dissolution rate and a combination thereof. Further bulk and tapped density of the polymorphic forms may also be evaluated.

The crystalline forms of Relugolix may be characterized by each of the above characteristics alone, and/or by all possible combinations, to distinguish from other polymorphic forms of Relugolix.

In another aspect of the invention, there is proved process for preparation of the crystalline Form-C2 of Relugolix.

Accordingly, in one process, the crystalline Form-C2 of Relugolix can be prepared by drying the crystalline Form-C1 of Relugolix for a sufficient time.

Preferably, the drying of Form-C1 of Relugolix is carried out at a temperature ranging from about 25° C. to about 120° C., preferably about 30° C. to about 100° C., more preferably about 35° C. to about 80° C.; preferably, for about an hour to about 48 hours, more preferably about 5 hours to about 40 hours, most preferably about 10 hours to about 35 hours.

In an embodiment, drying of the crystalline Form-C1 of Relugolix is carried out at a temperature of 45-55° C. for about 25-30 hours, to obtain crystalline Relugolix Form-C2.

According to alternate process embodiment, the invention provides a process for preparation of crystalline Form C2 of Relugolix which comprises at least two or more steps selected from;
a) Dissolving Relugolix in an organic solvent;
b) Optionally adding antisolvent to the solution of step a) followed by cooling to ambient temperature;
c) Optionally adding Relugolix at ambient temperature to obtain a solid; and
d) Drying the solid at a temperature of 30 to 55° C. to obtain crystalline Form C2 of Relugolix.

The organic solvent used for the dissolution of Relugolix is selected from the group consisting of N,N-dimethylacetamide (DMA), dimethylformamide (DMF), dimethylsulfoxide (DMSO), N-methylpyrrolidone (NMP), sulfolane, diglyme, 1,4-dioxane and the like; ether solvents such as methyl/-butyl ether, diisoproyl ether, tetrahydrofuran (THF) and the like; ester solvents such as methyl acetate, ethyl acetate, isopropyl acetate and the like; nitrile solvents such as acetonitrile, propionitrile and the like, ketone solvents such as acetone, methyl isobutyl ketone and the like; an aromatic hydrocarbons such as toluene, xylene and the like; aliphatic hydrocarbon solvents such as hexane, heptane and the like; alcohols such as Methanol, IPA, ethanol and water or a mixture thereof.

The optional anti solvent used is selected from the group consisting of water, protic polar solvents or mixtures thereof wherein protic polar solvents is preferably selected from C1-C5 alcoholic solvent such as methanol, ethanol, isopropanol, n-butanol, t-butanol and the like; aromatic alcohols, and mixtures thereof.

The Relugolix as used in step a) and step c) is selected from the group consisting of crude Relugolix, crystalline Relugolix Form C1 and Form C2 seed crystals of Relugolix.

Accordingly, in an embodiment, the Relugolix used in step a) of the process is crude Relugolix, prepared according to the methods reported in U.S. Pat. No. 7,300,935, which is incorporated herein by reference or crystalline Form C1 of Relugolix provided according to the example 1.

In another embodiment, the Relugolix used in step a) of the process is crystalline Form C2 of Relugolix prepared according to the example 2 of the present invention.

In yet another embodiment, the Relugolix used in step c) of the process is crude Relugolix, prepared according to the methods reported in U.S. Pat. No. 7,300,935, which is incorporated herein by reference.

In a further embodiment, the Relugolix used in step c) of the process is crystalline Form C2 of Relugolix prepared according to the examples 2 to 6 of the present invention.

Having described techniques best suited for producing distinct crystalline Forms C1 and C2 of Relugolix in a laboratory and industrial setting, those skilled in the art will appreciate that these forms may be accessible by yet other methods. The crystalline forms of the present invention may be prepared by dissolving, crystallizing, stirring, evaporating the solvent or seeding with crystal. The crystals may be isolated form the reaction mixture by any of the general techniques known in the art.

The novel polymorphic forms of Relugolix obtained according to the present invention are substantially free from other crystal and non-crystal forms of Relugolix. “Substantially free” from other forms of Relugolix shall be understood to mean that the polymorphs of Relugolix provided according to the present invention contain less than 10%, preferably less than 5%, of any other forms of Relugolix and less than 1% of other impurities, water or solvates. Thus, the crystalline forms C1 and C2 of Relugolix prepared according to the present invention contain less than 5% total impurities, preferably less than 3% total impurities. In a particularly preferred embodiment, the crystalline forms of Relugolix prepared according to the present invention contain less than 1% total impurities.

The processes of the invention may be used as a method for purifying any form of Relugolix, or salts thereof as well as for the preparation of the new polymorphic forms.

According to another aspect of the present invention, there is provided a pharmaceutical composition comprising polymorphic forms of Relugolix as described above, together with one or more pharmaceutically acceptable excipients. The Relugolix used in the preparation of pharmaceutical compositions may substantially consist of one of forms C1, or C2 described above, or may substantially consist of a combination of both the forms.

The pharmaceutically acceptable excipients are used to prepare variety of dosage forms of Relugolix Form C1 and Form C2 and may be selected from the group consisting of fillers, binders, diluents, polymers, distingrants, preservatives, sweetening agents, colors, flavors etc.

The Relugolix Form C1 or Form C2 can be formulated into various compositions and dosage forms by using suitable pharmaceutical excipients according to methods known in the art. These methods include blending the active and excipients and granulating the mixture using wet granulation or dry granulation that can be filled into hard gelatin capsule or these compacted granules may be compressed into a tablet.

In a preferred embodiment, the compositions comprise crystalline polymorphic Form C2 of Relugolix.

Alternately, a pharmaceutical composition of crystalline polymorphic Form C2 of Relugolix provided according to the invention may be formulated as an injection, which may be a viscous liquid solution, a clear solution or suspension. The injectable composition may contain one or more suitable solvents, to make the active in stable solution form. Along with suitable pharmaceutical solvents, pharmaceutical excipients such as buffers, antioxidants, solubilizers, etc. can also be added to the injectable composition.

According to yet another aspect of the present invention there is provided use of polymorphic Form C2 of Relugolix as described above, in the preparation of a medicament useful in treating or preventing diseases caused by gonadotropin releasing hormone (e.g., endometriosis, uterine fibroid and prostate cancer) in a patient in need of such treatment.

According to yet another aspect, the invention provides a method of treating diseases caused by gonadotropin releasing hormone (e.g., endometriosis, uterine fibroid and prostate cancer) in a patient in need of such treatment, which method comprises administering to the patient a therapeutically effective amount of crystalline Form C2 of Relugolix of Formula I or its pharmaceutically acceptable salts thereof, prepared by a process as described above.

The present invention will be described in more details in the following by way of examples, which are illustrative of further embodiments and shall not construe a limitation of the invention.

### EXAMPLES

### Example 1

### Process for the Preparation of Relugolix Form-C1

Relugolix (2 g) was dissolved in Dimethyl acetamide (4 ml) at 60-65° C. The clear solution was added to water (67 ml) over a period of about 5-10 min at 20-25° C. and stirred at same temperature for about 3 days. The crystals were collected by filtration and dried at 45 to 50° C., under reduced pressure until the weight became constant to give crystalline Form-C1 of Relugolix.

The isolated solid was identified as crystalline Form-C1 of Relugolix, by XRPD, TGA and DSC. (FIGS. 1, 2 and 3 respectively).

Yield: 1.5 gm

### Example 2

### Process for the Preparation of Relugolix Form-C2

Crystalline Form-C1 of Relugolix (0.2 g) was dried at about 45-55° C. for about 25-30 hours. The resulting solids were identified as crystalline Form-C2 of Relugolix, by XRPD, TGA and DSC (FIGS. 4, 5 and 6 respectively).

Yield: 0.16 g.

### Example 3

### Process for the Preparation of Relugolix Form C2:

Charged 290 gm of Relugolix and 580 ml of DMSO into a 1 L RBF. Stir the suspension for 20 min at 60° C. to get clear solution. Filtered the solution to remove any undissolved particulate through 0.45-micron filter and washed with 290 ml of DMSO. Charged 9.135 L of Water and 1.015 L of Ethanol in to a clean 15 L reactor and cool to 25° C. Added 15 gm of Relugolix Form C2 seed into the above reactor and stirred for 10 min. The above Product solution slowly added into the above reactor over a period of 10-15 min. Stir the reaction mass for 3.5 hrs at RT. Filter the reaction mass and suck dry for 20 min. wet weight: 321 gm.

Dry the above suck dried material under vacuum at 45° C. for 6-8 hrs, resulted the titled compound. The XRPD, TGA and DSC of this example matches with FIGS. 4, 5 and 6 respectively.

Yield: 260 gm.

### Example 4

### Process for the Preparation of Relugolix Form C2:

Charged 6 gm of Relugolix Form C2 seed material and 2.54 L of water in to the 5 L reactor. Stir for 5-10 min to get uniform seed suspension. To the above seed slurry charged 127 gm of Relugolix Crude and stir at 23° C. for 3 hrs. Then slowly added 260 ml of IPA into the above reaction mass and stir at 23° C. for 1 hr. Filter the reaction mass through PNF and kept under 1-2 Kg of Nitrogen pressure for 2 hrs. Wet weight: 160 gm.

Dry the above suck dried material under vacuum at 30° C.-40° C. for 40 hrs, resulted the title compound. The XRPD, TGA and DSC of this example matches with FIGS. 4, 5 and 6 respectively.

Yield: 115 gm.

### Example 5

### Process for the Preparation of Relugolix Form C2:

Charged 135 ml of Water and 15 ml of Methanol and 750 mg of Relugolix Form C2 seed material into a clean RBF. Stir for 10-15 min to get the uniform slurry. Charged 15 gm of Relugolix crude into the above slurry. Stir for 10-15 hrs at 20-25° C. Filter the reaction mass and suck dry for 30 min. The resulted material and dried under vacuum at 40-50° C. for 10-15 hr to get the titled compound. The XRPD, TGA and DSC of this example matches with FIGS. 4, 5 and 6 respectively.

Yield: 12.7 gm.

### Example 6

### Process for the Preparation of Relugolix Form C2:

Charged 135 ml of Water and 15 ml of Methanol into a clean RBF. Charged 15 gm of Relugolix crude into the above RBF. Stir for 10-15 hrs at 20-25° C. Filter the reaction mass and suck dry for 30 min. The resulted material and dried under vacuum at 40-50° C. for 10-15 hr to get the titled compound. The XRPD, TGA and DSC of this example matches with FIGS. 4, 5 and 6 respectively.

Yield: 12.7 gm.

1. A Crystalline Form C-2 of Relugolix, characterized by data selected from one or more of the following:
a) an X-ray powder diffraction pattern substantially as depicted in FIG. 4;
b) an X-ray powder diffraction spectrum comprising peaks at 6.87, 9.30, 18.63, 19.97 and 22.78±0.2° 2θ;
c) a TGA thermogram substantially as depicted in FIG. 5;
d) TGA thermogram characterized by a weight loss of approximately 0.152% at temperature about 175° C.; and
e) combinations of the above data.

2. The crystalline Form C-2 of Relugolix, as claimed in claim 1, further characterized by X-ray powder diffraction having peaks at 16.09, 21.34, 24.61 and 26.40±0.2° 2θ.

3. The crystalline Form C2 of Relugolix, as claimed in claim 1, characterized by a DSC thermogram as shown in FIG. 6.

4. The crystalline Form C2 of Relugolix, as claimed in claim 3, characterized by a DSC thermogram having a single endotherm peak melting with an onset at 194.60±5° C. and a peak maximum at 197.37±5° C.

5. A process for preparation of crystalline Form C2 of Relugolix comprising at least two or more steps selected from;
a) Dissolving Relugolix in an organic solvent;
b) Optionally adding antisolvent to the solution of step a) followed by cooling to ambient temperature;
c) Optionally adding Relugolix at ambient temperature to obtain a solid; and
d) Drying the solid at a temperature of 30 to 55° C. to obtain crystalline Form C2 of Relugolix.

6. The process as claimed in claim 5, wherein, the organic solvent used for the dissolution of Relugolix is selected from the group consisting of N,N-dimethylacetamide (DMA), dimethylformamide (DMF), dimethylsulfoxide (DMSO), N-methylpyrrolidone (NMP), sulfolane, diglyme, 1,4-dioxane and the like; ether solvents such as methyl/-butyl ether, diisoproyl ether, tetrahydrofuran (THF) and the like; ester solvents such as methyl acetate, ethyl acetate, isopropyl acetate and the like; nitrile solvents such as acetonitrile, propionitrile and the like, ketone solvents such as acetone, methyl isobutyl ketone and the like; an aromatic hydrocarbons such as toluene, xylene and the like; aliphatic hydrocarbon solvents such as hexane, heptane and the like; alcohols such as Methanol, IPA, ethanol and water or a mixture thereof.

7. The process as claimed in claim 5, wherein, the optional anti solvent used is selected from the group consisting of water, protic polar solvents or mixtures thereof wherein protic polar solvents is preferably selected from C1-C5 alcoholic solvent such as methanol, ethanol, isopropanol, n-butanol, t-butanol and the like; aromatic alcohols, and mixtures thereof.

8. The process as claimed in claim 5, wherein, the Relugolix used in step a) is selected from the group consisting of crude Relugolix, crystalline Form C1 of Relugolix and Form C2 seed crystals of Relugolix.

9. The process as claimed in claim 5, wherein, the Relugolix used in step c) is selected from the group consisting of crude Relugolix and Form C2 seed crystals of Relugolix.

10. The process as claimed in claim 5, further comprising a step of drying of the crystalline Form-C1 of Relugolix is carried out at a temperature of 45-55° C. for about 25-30 hours, to obtain crystalline Relugolix Form-C2.

11. A pharmaceutical composition comprising crystalline Form C2 of Relugolix or its pharmaceutically acceptable salts thereof in association with one or more suitable pharmaceutical excipients or carriers, to obtain in desired dosage form.

12. The pharmaceutical composition as claimed in claim 11, wherein, the dosage form is selected from the group consisting of tablets, powders, granulates, capsules, sachets, aggregates, suppositories, troches, and lozenges, as well as liquid syrups, suspensions, and elixirs.

13. The pharmaceutical composition as claimed in claim 11, wherein the composition is obtained by combining the crystalline Form C2 of Relugolix, with at least one pharmaceutically acceptable excipient, to obtain the formulation in desired dosage form.

14. The crystalline Form C2 of Relugolix or its pharmaceutically acceptable salts thereof, as claimed in claim 1 for use in the manufacture of a medicament for treating diseases caused by gonadotropin releasing hormone (e.g., endometriosis, uterine fibroid and prostate cancer).

15. (canceled)
